(19) United States
(12) Patent Application Publication
(10) Pub. No.: US 2005/0181076 A1
(43) Pub. Date: Aug. 18, 2005

(54) Compositions and methods for treatment of diabetes

(76) Inventor: Randy Ziegler, Newport Coast, CA (US)

Correspondence Address: Stefan J Kirchanski Esq;Liner Yankelevitz, Sunshine & Regenstreif LLP, 1100 Glendon Avenue 14th floor, Los Angeles, CA, 90024, US

(21) Appl. No.: 10/863,459

(22) Filed: Jun. 7, 2004

Related U.S. Application Data
(62) Division of application No. 09/967,030, filed on Sep. 27, 2001, now abandoned.
Application No. 09/967,030 is a continuation-in-part of application No. PCT/US00/08957, filed on Apr. 4, 2000.
(60) Provisional application No. 60/127,824, filed on Apr. 5, 1999.

(51) Int. Cl. A61K 31/7048; A61K 31/353; A61K 35/78

(52) U.S. Cl. 424/725; 514/27; 514/456

(57) ABSTRACT

Flavonoids, especially luteolin, are shown to be effective against insulin dependent (Type I) and insulin independent (Type II) diabetes mellitus. It is demonstrated that luteolin works in mammals by binding and blocking the Kᵥ1.3 potassium channel of T-cell and Beta cells. Antidiabetic and anti-autoimmune compounds can be selected by measuring their ability to bind to and block the Kᵥ1.3 channel.

The present application is a Continuation-in-part of PCT/US00/08957 which designates the United States and was filed on Apr. 4, 2000 which in turn was based on and claimed priority from Provisional Application Serial No. 60/127,824, entitled “COMPOSITIONS, PRODUCTS, AND METHODS FOR TREATMENT OF DIABETES” which was filed on Apr. 4, 1999 and which is incorporated herein by reference.

### BACKGROUND OF THE INVENTION

1. Field of the Invention

The present application concerns the field of natural products and more specifically plant extracts and compounds useful for the treatment of diabetes.

2. Description of Related Art

Diabetes mellitus (honey or sugar diabetes) a potentially devastating, complex disorder of glucose metabolism, which is currently partially controllable by insulin injections and/or drugs, is increasing in worldwide frequency. In the United States over ten million persons are estimated to have diabetes. The financial cost is in the many billions of dollars reflecting treatment expense and loss of productivity while the human cost in impaired function, progression to blindness, limb amputations, kidney failure and heart and vascular disease is immeasurable.

While the hallmark of diabetes is high blood sugar with concomitant excretion of sugar in the urine, the disease has two major variants:
Type I or Juvenile Onset (Insulin Dependant Diabetes Mellitus—IDDM); and
Type II or Adult Onset (Non-insulin Dependant Diabetes Mellitus—NDDM).
   These variations are named for the approximate time of onset, but onset time is not actually determinative. In a nutshell IDDM appears to be an immune modulated version of the disease in which insulin production is impaired whereas NDDM is a disorder in which the cells fail to respond to insulin.

Diabetes is recognized in the ancient literature of Egypt, China, and India. Johann Conrad Brunner made the first suggestion that diabetes might involve a pancreatic disorder in 1682. It was not until the 20th Century, however, that the diabetic condition was clearly associated with insulin—either the formation and secretion of insulin by the pancreas or the influence of circulating insulin on the cells of the body.

The simple sugar glucose is a primary energy source for human cells Glucose is required for optimal growth, development, and for maintenance of the central nervous system. The brain is an avid consumer of glucose such that any significant lowering of blood glucose results in a concomitant drop in the glucose level in the brain with resulting cessation of normal brain function (coma). The entry of glucose into the cells and the metabolism of the glucose within the cells are critical to sustain life in the human body. Insulin, a regulatory transport hormone, controls the uptake and transport of glucose into the cells either for energy production or for storage therein. Glucose enters the bloodstream from the digestive system. If the intracellular level of glucose is too low or the blood level of glucose is too high, insulin is released to mediate the uptake of glucose by the cells for metabolism or storage, respectively. If the blood level of glucose is too low, other hormones mediate the release of glucose from glycogen (a starch-like storage polymer). Thus, insulin is necessary for the glucose homeostasis found in proper body metabolism. The proper concentration of insulin in the blood is critical. A lack of insulin leads to coma and death from metabolic problems caused by excessive blood sugar. On the other hand, an excess of insulin results in shock caused by excessively low blood sugar. Similarly, if the cells fail to respond properly to insulin, the homeostasis is disrupted and excessive blood sugar levels result.

When blood sugar is uncontrolled serious metabolic imbalances ensue-elevated glucose levels lead to ketosis and to damaging alterations in blood pH while inadequate glucose levels lead to lethargy and coma. Diet drugs and/or and periodic injections of insulin are now used in an attempt to control life-threatening swings in blood glucose. It is now well established that the damage is caused by excessive glucose and not directly by lack of insulin. Excess glucose combines with hundreds of proteins essential for normal metabolism and in that way damages the cellular machinery of the body.

Excess blood glucose is responsible for many of the morbidity of diabetes. Diabetics often suffer from small blood vessel disease (microangiopathy) caused by the thickening of the walls of the capillaries over time. As a secondary result, capillaries become leaky, leading to retinopathy and nephropathy. In common terms, diabetes leads to blindness and kidney damage. In addition, hardening of arteries in the body may also cause premature coronary rupture. Neuropathy also occurs in diabetics and causes the loss of feeling in the lower extremities. Gangrene and subsequent amputation are common occurrences resulting from diabetes mediated vascular deterioration.

Insulin is produced within the pancreas by 1.5 million beta cells located in clusters known as the Islets of Langerhans. Insulin is a moderate sized protein composed of two chains: an alpha chain of 21 amino acids and a beta chain of 30 amino acids linked to one another by disulfide bonds.

There are many theories for explaining the impairment of insulin production by the pancreas that leads to the diabetic condition. Reference is made to a paper entitled “Autoimmune Imbalance and Double Negative T Cells Associated with Resistant, Prone and Diabetic Animals”, Hosszufalusi, N., Chan, E., Granger, G., and Charles, M.; J Autoimmun, 5: 305-18 (1992). This paper shows that inflammation of the pancreatic Islets interrupts insulin production. Specifically, the insulin producing beta cells in the pancreatic islets are destroyed by immune attack. Such beta cell destruction is recognized as being due to attack by several types of immune cells including NK (natural killer) cells and double negative (CD4⁻[W3/25+OX19+]/CD8⁻[OX8+OX19+]) T-Lymphocytes.

Further research progress in this area has been achieved and reference is made to a paper entitled “Quantitative Phenotypic and Functional Analyses of Islet Immune Cell Before and After Diabetes Onset in the BB Rat”, Hosszufalusi, N., et al., Diabetologia 36: 1146-1154 (1993), where it was demonstrated that double negative T cells (CD4⁻/CD8⁻, double negative cells) increased to about 30 percent of the islet T-cell population at the onset of diabetes. The cytolytic behavior of these cells was shown to be tissue specific for Islet cells.

A paper entitled “Clonal deletion and autoreactivity in extrathymic CD4/CD8⁻ (double negative) T cell receptor-alpha/beta T cells”, Prud'homme, G. J., Bocarro, D. C., et al., J Immunol. 147: 3314-8 (1991), discusses the suppression of known variable region gene VB 16 and the associated cytokines, by a blocking compound which corrects the metabolic imbalance that results in autoreactive double negative T-cells-cells that cause inflammation of the Islets in the pancreas. A corrective balance of cell types was proposed as follows: B-cells>T-cells (CD4>double negative>CD8)>NK cells>macrophages. It is also recognized that the autoimmune response results in macrophage activation by the double negative T-cells, wherein activated macrophages then attack body cells. When proper depletion of T-cell clones in the thymus fails, double negative T-cells escape and become potentially autoreactive clones. It has been theorized that the CD8 protein, expressed by the majority of NK cells, can be modulated by administration of monoclonal antibodies to reduce the incidence of diabetes. The administration of polyclonal antibodies directed towards the NK cell glycolipid AGMI also prevents autoimmune Islet destruction.

On the neurological level, it is believed that aldosterone, from the adrenal cortex, sets in motion a set of reactions at the surface of all cells of body tissues to regulate the uptake and retention of sodium and to extrude potassium. Lowered serum sodium and the high serum potassium levels enhance aldosterone secretion. The adrenal glands are influenced by the neurotransmitter dopamine, an adrenal suppressor and by the neurotransmitter seratonin, an adrenal stimulator; low potassium levels impact dopamine production and, therefore, alter aldosterone and cortisol secretion. In addition, other factors are involved in the negative feedback of pituitary corticotropin to cortisol. These factors have been recognized as atrial natriuretic peptides, or sodium excreting hormones, that inhibit secretion of aldosterone, sodium chloride, potassium, and phosphorous. It has also been recognized that there is an interference with the ongoing inhibition of prolactin by dopamine from the hypothalamus as can be seen during the invasion of the pituitary stalk by pineal tumors. These factors may be involved in the immune abnormalities leading to insulin dependent diabetes or in the abnormal insulin responses of insulin independent diabetes.

In a paper entitled “Auto Immune Diseases Linked to Abnormal K+ Channel Expression in DN CD4⁻ and CD8⁻ T cells”, Chandy, K. G., et al., Eur. J. Immunol. 20: 747-751 (1990), the impact of potassium on the cytotoxicity created by DN T-cells is discussed. Similarly bioamines and neuropeptides were found to function as neurotransmitters to neuromodulate the inhibition or stimulation of neurotransmission i.e. opioid peptides. In such mechanisms, the hypothalmous synthesizes and secretes neurohormones directly from and through the nerve axon to a capillary network transported through the hypophyseal portal circulation to the anterior pituitary gland.

A paper entitled “Role of growth factors in pancreatic cancer”, Korc, M., Surg Oncol Clin N Am., 7: 25-41 (1998), explains how insulin stimulates growth and cell proliferation through a tyrosine kinase dependent pathway. Insulin, like growth factor I (RGF-I), is a mitogenic polypeptide that regulates cell cycle progression. IGF-I and insulin are heterotetrameric proteins that possess intrinsic tyrosine kinase activity. IGF-I actions are dependent upon binding to its own specific cell surface receptors. Both insulin and IGF-I activate insulin receptor substrate −I(IRS-1), an important multisite docking protein implicated in mytogenic signaling. Activation of mytogenic pathways is magnified as a consequence of mutations in the K-ras oncogene and cell cycle associated kinases, such as p16. Insulin exerts mytogenic effects on cells by activating the IGF-I receptor, which leads to phosphorylation of IRS-1, an important regulatory protein that mediates the growth promoting effects of insulin. The tyrosine kinases are thought to be truncating the sequence of production of dopamine so that a post receptor defect is caused which has no affinity for the necessary glucocorticoid, but has affinity for the DN (double negative) T-cell CD4⁻ and CD8⁻ proteins. It is theorized that this could be altered by proteoglycin to rebalance the K+ (potassium) channel to allow a gate voltage to buildup and permit secretion of adequate amounts of aldosterone. It was also believed that a valance corrected aggregated series of polypeptides assimilated into a proteoglycan would accomplish this result.

Diabetes is considered to be insidious, since there is no cure known at this time. Various treatments, however, have been used to ameliorate diabetes. For example, dietetic measures have been employed to balance the relative amounts of proteins, fats, and carbohydrates in a patient. In addition, diabetic conditions of moderate or severe intensity are treated by the administration of insulin. Also, prescription drugs such as “Glucoside” have been employed to rejuvenate impaired insulin production in adult onset diabetics. Other drugs are used to modulate the effectiveness of insulin. In any case, treatment of diabetes, of either juvenile or adult onset types, have achieved only partial success.

### SUMMARY OF THE INVENTION

In accordance with the present invention a novel and useful composition for treating diabetes is provided.

The treatment of the present invention was discovered because the inventor found that a steam or aqueous extract of a plant known as Brickellia californica was effective in controlling blood sugar. For use plant is gathered, dried, and combined with boiling water. The extract is then taken orally by a patient on a periodic basis. The genus Brickellia is known to be rich in flavonoids and other secondary plant products. The genus is large and many species are mentioned in folk medicine including, besides B. californica, B. ambigens, B. arguta, B. brachyphylla, B. cylindracea, B. eupatoriodes, B. glutinosa, B. grandiflora, B. laciniata, B. lemmonii, B. oblongifolia, and B. veronicaefolia. Other species of the genus appear to have some or all of the active components of B. californica.

Specific flavonoids have been extracted and fractionated from Brickellia californica and administered to diabetics with results similar to those produced by the extract. The flavonoids specifically used were dihydrokaemferol and apigenin, a flavone. It was then discovered that these flavonoids are most effective in combination. Moreover other Brickellia flavonoids, specifically myricetin and especially luteolin, have been determined to be effective in treating diabetes alone or in combination, or in combination with dihydrokaemferol and apigenin. What was truly surprising was the discovery that luteolin, in particular, is effective in lowering the blood sugar and generally alleviating diabetic symptoms in IDDM as well as NDDM. This result was unexpected because conventional wisdom teaches that these two forms of diabetes have basically different causes. I have discovered an underlying “molecular switch” that controls both forms of diabetes. This “switch” can be operated by luteolin and similar flavonoids.

### BRIEF DESCRIPTION OF THE FIGURES

FIG. 1 shows the 34-day drop in blood sugar in a Type I human diabetic in response to daily administration of luteolin.

FIG. 2 shows the range of blood sugar in a Type II human diabetic (KT) over one week.

FIG. 3 shows the drop of blood sugar in the diabetic of FIG. 2 following administration of 350 mg of luteolin.

FIG. 4 shows responses in the blood sugar of a Type II human diabetic (TC) to 350 mg luteolin (measurements made in duplicate).

FIG. 5 shows the long term response of Type II diabetic rats to administration of luteolin.

### DETAILED DESCRIPTION OF THE PREFERRED EMBODIMENTS

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide treatment of both insulin-dependent and non-insulin dependent diabetes through the administration of flavonoids-particularly through the administration of luteolin.

Luteolin is a natural molecule found in historical floods such as artichokes, grapes, apples, millet corn and plants such as Brickellia californica. The molecule is usually synthesized by plants from transcinnamic acid and is classified as a flavonoid, one of nearly four thousand known flavonoids. Luteolin is can be used by plants as a molecular signaling molecule which stimulates and or suppresses gene expression. The luteolin molecule is comprised of two phenyl rings, A and B, and a pyran ring, C ring. The pyran, C ring is abutted to the A (phenyl) ring and forms a double bond at the 4 and 9 positions in a planar configuration. The third ring, or B ring, is attached to the C ring at the 2 position of the C ring by a single bond with a 23-½ degree twist. The pyran ring has an oxygen in the ring at the one position and a carbonyl between the 3 and 4 positions of the conjugated rings A and C. The A ring is hydoxylated at positions 5 and 7 while the B ring is hydoxylated at 3′ and 4′ positions. Between positions 2 and 3 is a double bond. I have found that it is this double bond open at the 3 position that is critical to allow the delta positive of the molecule to exert its effect.

Rutin is a luteolin glycoside with an -0-Sugar at the 3 position. Rutin is found in eucalyptus leaves and many flowers; however rutin has no hypoglycemic effect but does scavenge free radicals and is used to slow down cataract formation and macular degeneration. This indicates that the flavonoid effects on cataracts is separate from the effects of luteolin and that luteolin glycosides are not active hypoglycemically. Hervwig Bucholtz of Merck GmbH, has developed a synthesis for luteolin from rutin by removing the -0-Sugar at the 3 position with NaOH and sodium dithionate. Luteolin is however hypoglycemic showing therefore the 3 position is absolutely essential for the desired effect of lowering blood sugar in the diabetic. Luteolin has a delta positive charge exerted at the 3 position allowing bonding to other compounds (sugars) by means of an oxygen linkage. The molecule ionically attracts the hex ringed sugars and penta ringed sugars by its delta positive charge. Luteolin has several measured and observable biological effects.

Luteolin is a ligand to Iodothreonine Deiodinase, an oxygen transport hormone. By inhibiting this hormone, oxygen transport through the mitochondrial wall is slowed, thereby inhibiting the production of ATP from ADP and ATP synthase. Further, the pyran oxygen and carbonyl are end terminus electron acceptors. Therefore the electron gradient is slowed by sequestration of the hydrogen ions used in the electron transport chain of NAD to NADH and FAD to FADH and throughout the mitochondrial wall. This slows the pumping of the electrons to ADP and ATP synthase for ATP formation. When ATP formation is inhibited, mitochondrial respiration does not produce H₂O₂ as a byproduct. H₂O₂ stimulates the tyrosine kinases 394 and 505 in the proto-onco gene p56lck,. See, “The Activated Form of the Lck Tyrosine Protein Kinase in Cells Exposed to Hydrogen Peroxide Is Phosphorylated at Both Try-394 and Tyr-505” by Hardwick and Sefton JBC Volume 272, number 41 Oct. 19, 1997 pp. 25429-25432 (which publication is specifically incorporated herein by reference). A gene, p56Lck is the signal transducer necessary for the proliferation of CD4⁻ and CD8⁻ T Cells. These are the T Cells that cause diabetes. See attached paper “Quantitative Analysis Comparing All Major Spleen Cell Phenotypes in BB and Normal Rats: Autoimmune Imbalance and Double Negative T Cells Associated with Resistant, Prone and Diabetic Animals” by Dr. M. A. Charles et. al., Journal of Autoimmunity, 1992, Vol 5, pp 305-319, (which paper is specifically incorporated herein by reference. These T- cells escape the thymic deletion process and are autoreactive. This causes inflammation of the pancreatic Beta cell walls causing the inhibition of insulin release. Luteolin scavenges free radical, see the paper “The Effects of Plant Flavonoids on Mammalian Cells: Implications for Inflammation, Heart Disease, and Cancer” by E. Middleton et. al., Pharmacological Reviews, Vol. 52, No 4, pp. 673-751, 2000 (which publication is specifically incorporated herein by reference). Certain flavonoids can do this with the 3″ and 4″ hydroxyl groups on the B ring and 5 and 7 hydroxyl groups on the A ring and pyran oxygen and carbonyl on the C ring. Then as H₂O₂, O₂⁻, OH⁻ are bonded and absorbed out of the loop, then tyrosine kinases are not activated and T Cell proliferation does not ensue. Pancreatic Beta Cells are not inflamed and insulin is released normally.

Oxygen transport is inhibited by luteolin action on Iodothreonine Deiodinase and conversion of ADP to ATP is slowed down not allowing these CD4⁻/CD8⁻ cells to be activated. Research has shown that Mg₂⁺ is the causal effector in the production of these dangerous T cells. If these ions are chelated, the catalytic production of ATP is inhibited, electron transport and the linked oxidation of glucose is inhibited. Also, Cu₂⁺ copper is sequestered in the liver, stopping the fragmentation of and modification of LDL (Low Density Lipoprotein). This prevents the copper catalysis and O₂⁻ binding that creates aldehydes and the alcoholic sugars such as sorbitol. These alcohols degrade the collagen matrix in the eye leading to retinopathy by leaving collagen stripped of protein when exposed to UV damage. Cataracts then occur as a protection to the damaged and degraded retina or through a direct reaction of the aldehydes and alcohols on the protein of the lens. Metal binding abilities, similar to those of biguanides, chelate Cu₂⁺ ions to stopping the catalytic breakdown of glycogen in the liver. This prevents “sugar dumping” or glucogenesis from the starch stored in the liver. By chelating the ions in the catalytic pathway the diabetic can level out his spiking and the following neural exhaustion. This creates a carbohydrate deficit and the need for intake of a sugar and thus a spike due to exhaustion of stored glucose polymers.

This absorption necessitates the demand for insulin on an organ already performing poorly and under immunological attack by the CD8⁻ Natural Killer cells. Certain flavonoids stop this pathway by sequestering O⁻ from the lipid peroxidation cycle thus shunting fragmentation of cell membranes and piped byproducts that engender LDLs. Luteolin binds also combines with another element-Nitrogen. Nitric oxide is formed between smooth muscle and endothelial cells and gives a byproduct of H₂O₂. By stopping nitric oxide formation, NO, the main signal transducer for premeditation of a heart attack is stopped and is mitigated in the formative steps by oxygen scavenging and nitrogen bonding. Nitrogen bonds to the carbonyl and pyran oxygens to form NO. By stopping lipid peroxidation due to free radicals, beta cells that are exquisitely sensitive to oxidative damage due to poor enzymatic defense are protected. If esterification of a fatty acid at the cell wall ensues, then production of PLA₂ ensues, which further exacerbates the constellation of modalities leading to the state of diabetes. This further inflames the Beta cell wall. PLA₂ leads to the production of CD8⁻ Natural Killer cells, to abate and mitigate aberrant cells. It is the fortuitous crossing of CD4⁻ and CD8⁻ at cystein that signals calmodulin and Kᵥ1.3 to open and begin proliferation of the T-Cells leading to the diabetic state of siege.

When the toxic CD8⁻ Natural Killer Cells combines with the CD4⁻ Helper T Cells at cystein they electronically stimulate calmodulin. This voltage sensor activates one of the 80+ super gene channels necessary for the activation of the CD4⁻ and CD8⁻ T Cells, Kᵥ1.3 a voltage gated potassium channel. If this channel is not activated by calmodulin the T-Cells remain in their resting states. Promulgation of the diabetic causalities and effectors does not ensue. Luteolin blocks this channel as discovered recently by patch clamp analysis by the Electrophysiology Department at the University of California, Irvine. There are 200 pores in a resting Beta cell. When cell potential reaches 1.3 nVolts, the Kᵥ1.3, voltage gated potassium channel opens to expose the tyrosine kinase tails. These kinases when stimulated turn on the ras-Oncogene, a cancer promoter, which turns on Protein Kinase C, another tumor promoter. These drive the Nuclear Factors of the Activated T-Cell, such as cAMP; which stimulates the susceptibility genes associated with diabetes such as those on chromosome 19q13.3. These in turn produce InterLeukin-2, an inflammatory cytokine messenger signaling further T-Cell proliferation. When CD8⁻ cells sample the external receptors of the Beta cell, they find and bind to laminin to sites, such as AGM1, and releases InterLeukin-2 upon calcium loading. This inflammatory cytokine causes cell activation and suppression of insulin release. By stopping ATP production, and H₂O₂ as its byproduct in these cells, in both Beta cells and CD8⁻ cells these cells are left in a resting state, Beta cell attacks are quelled, and Beta cells are able to release insulin when sensitized by glucose. The voltage sensor calmodulin sense the delta positive in glucose when it reaches the Beta cell wall and insulin should be released. But a secondary set of reactions also occur if left unregulated. Esterification of the fatty acids in the cell wall, of the Beta cell occurs. Upon phosphorylation Phospholipase A₂ is produced and Protein Kinase C is stimulated. These are byproducts of the Arachidonic Acid cascade and signal tumor promotion by PKC and a lipase production that is inflammatory in the cell wall, further exacerbating Beta cell inflammation and compounding the problem of the Beta Cell inhibition of the release of insulin.

Further consequences of Arachidonic Acid activation are the production of Lipo-oxygenase cytokines such as Prostaglandins and Thromboxanes. These cause heart attacks and organ failure. Simultaneously, Cyclo-oxygenase products are produced such as the Leukotrienes and HETE (hydroeicosanoic tetraeinaic acids) families of molecules. These cytokines, specifically 5-HETE and 12-HETE damage genetic products and lead to altered gene expression. Epoxide diols can form in the DNA leading to strand damage. These can cause frame shift mutations by altering nucleic acid sequences leading to genetic diseases. Uracil is used twice to code for tyrosine. Uracil has a pyrimidine base on a sugar with a phosphate base attached to the nucleic strand. Hydrogen bonding occurs between complimentary base pairing. Free radicals and inflammatory cytokines can damage and break this bonding leading to improper codon sequencing and ribosome misconstruction. Transcripts are transcribed now with misinformation. This stimulates onco-gene expression and the proliferation of CD8⁻ NK Cells.

The Calcium Release Activated Calcium channel is a small conductance channel that releases calcium and ATP-ases when not blocked by a regulatory voltage gate, or molecule. It is this slow release that causes the diabetic to never reach the threshold of Kᵥ1.7 for release of insulin. Further complications ensue when glucose spurs ATP to be released prematurely. It is the overproduction of ATP that causes CD4⁻/CD8⁻ cells to be stimulated.

Glucose stimulates the production of ATP and hence the byproduct of H₂O₂ and therefore the byproduct of CD4⁻/CD8⁻ T Cells, and Phospholipase A2. Glucose is immediately processed and is the only fuel for the brain. However it is not released slowly as in fruit or vegetables being that they are fiberous and release their sugars slowly and in a controlled fashion. The overly rapid production of ATP, and hence its byproduct H₂O₂ from the mitochondria, and Phospholipase A₂ perpetuate and promulgate the diabetic maelstrom.

All of these cycles are calcium driven. If calcium is sequestered at the cell surface, then potassium is not pumped out and ATP is not released. Then Kᵥ1.7 can be activated when the proper potential is reached, so that insulin will be released from the Beta cell. All of the inflammatory cytokines can be pre-empted and a rapid achievement of the electronic force achieved to release insulin. Luteolin sequesters calcium by means of its hydroxyl groups on the distant polar ends of the flavonoid which have negative charges. An electronic cloud, by reason of the 23-½° twist of the B ring chelates calcium. Further Van Der Waals attractions are enhanced by the regional proximity of the hydroxyl groups, 3′ and 4′ on the B ring, and 5 and 7 on the A ring to the pyran oxygen, and, carbonyl, between the 3 and 4 positions of the planar conjugated rings. Additional strength is garnered from the desire of the pyran and carbons wanting to accept electrons and drawing a charge so that calcium is netted by the entire molecule, since oxygen is an end terminus electron acceptor. The 23-½ twist atomically provides the overall net for the calcium Ca₂⁺ cation. Calcium being now held at the cell surface, Kᵥ1.3 is blocked, electronically so that the potassium gradient builds to hyperpolarize thus reaching Kᵥ1.7, the insulin releasing channel. It has now been discovered that luteolin penetrates into the pore of Kᵥ1.3 possibly having a direct effect on the critical tyrosine residues preventing their activation. In this case Calmodulin would not be able to pump the cell to Kᵥ1.3 allowing a hyperpolarization to Kᵥ1.7. Kᵥ1.3 has a 6 amino acids long transmembrane region that has been sequenced. The natural resting state potential of the Beta cell is −20 nV. When luteolin was tested at 100 nM, the cell remained in its resting state and Kᵥ1.3 was blocked completely. When the cell reached +30-50 nV Kᵥ1.7 activates and opens some 600 pores and released insulin.

This explanation is presented to explain the incredible and unexpected effectiveness of luteolin in the treatment of both insulin dependent (Type I) and insulin independent (Type II) diabetes. Insulin dependent diabetes has long been known to be an autoimmune disease. It is perhaps not too surprising that T-Cell inhibition by luteolin (as detailed above) could modulate or prevent the autoimmune reaction leading to Type I disease. At first look it might seem surprising that luteolin would show an effect on established Type I diabetics. Conventional wisdom indicated that all of the Beta cells in such a diabetic had been destroyed. However, more recent experiments using powerful antineoplastic agents to interfere with the immune system have shown that in many if not most cases of insulin dependent diabetes the autoimmune assault on the Beta cells is an ongoing process. That is a residual population of Beta cells exists but are prevented from releasing insulin due to the continued immune attack on the cells. Under such a scenario the anti-inflammatory effects of luteolin might be expected to rescue these Beta cells and allow them to function more normally. This is probably the case. However, what is even more exciting is my discovery that luteolin directly affects Kᵥ1.3.

It appears that Kᵥ1.3 is central to a series of processes, detailed above, which lead to failure of insulin release under hyperglycemic conditions in certain individuals. That is, excess glucose leads to a cascade of biochemical interactions that culminate in Kᵥ1.3 failing to allow the cells to reach sufficient potential to allow Kᵥ1.7 controlled release of insulin. I believe I am the first to conceive and show that Kᵥ1.3 is the central switch for diabetes. When luteolin or similar effectors enter and bind to this molecule autoimmune inflammatory processes are prevented (essentially prevention of Type I diabetes) and hyperglycemic blocking of insulin release is prevented (essentially control of Type II diabetes). Although my present preferred modulator of the Kᵥ1.3 “diabetes switch” luteolin, other molecules that bind to and block Kᵥ1.3 are certainly within the bounds of my invention. To recap I have discovered that Kᵥ1.3 is a central molecule in the disease of sugar diabetes. This switch operates in two manners. First, it quenches the T-Cell stimulation required for autoimmune attack on Beta Cells. I have also discovered that this autoimmune modulation by molecules that bind to Kᵥ1.3 are important in other autoimmune diseases. Second, molecules, such as luteolin, that bind to Kᵥ1.3 directly block the hyperglycemic blocking of insulin release found in Type II diabetics. Undoubtedly both of these effects are involved in the ameliorating effect on Type I diabetes shown by luteolin and similar Kᵥ1.3 binding molecules.

Previously there has been some indication that flavonoids might show hypoglycemic properties. My invention shows that this property is due to binding to Kᵥ1.3 and that, therefore, flavonoids and other compounds can be screened for hypoglycemic potential by measuring their effects on Kᵥ1.3.

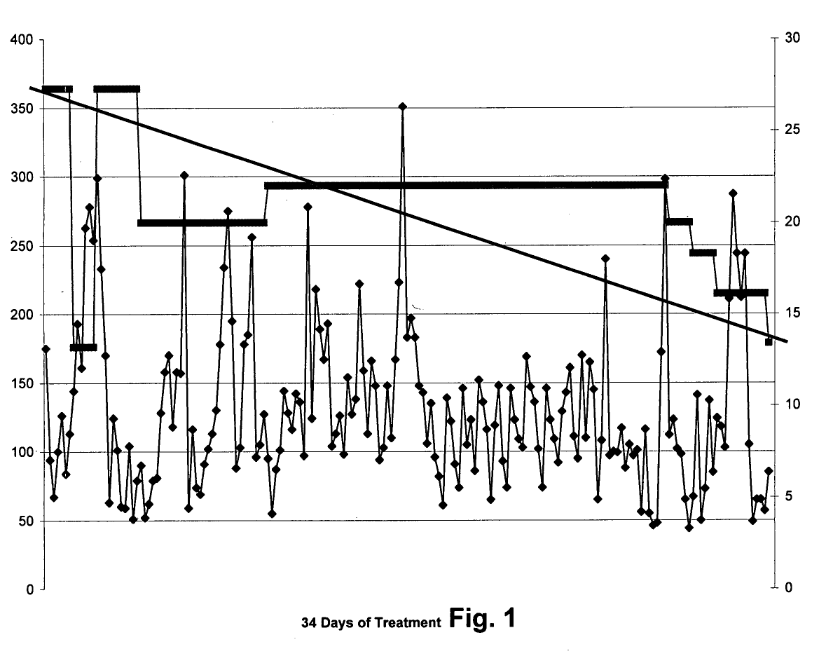

LW is a Type I insulin dependant female since 13, on a MiniMed pump for 10 years. She is approximately 34 years old. Upon receiving 150 mgs, scaled down to 20 mg of luteolin per day, she decreased her use of insulin by 50% in 34 days. An immediate initial reduction of 50% of required insulin use was seen after the first dose of luteolin. LW took her pump off at night during the 4th week of experimentation. Doses were dropped on the 2nd and successive doses to maintain a controlled linear progression. LW went from 27 units of insulin per day to a PK (PharmacoKinetic) dosage of 25 mgs, and 13.5 units of insulin per day. This shown graphically in FIG. 1 where the thicker horizontal line represents insulin dosage in mg (left scale). The diagonal line represents the overall drop in blood sugar (right scale) over the 34 days from about 350 mg/dl to about 200 mg/dl.

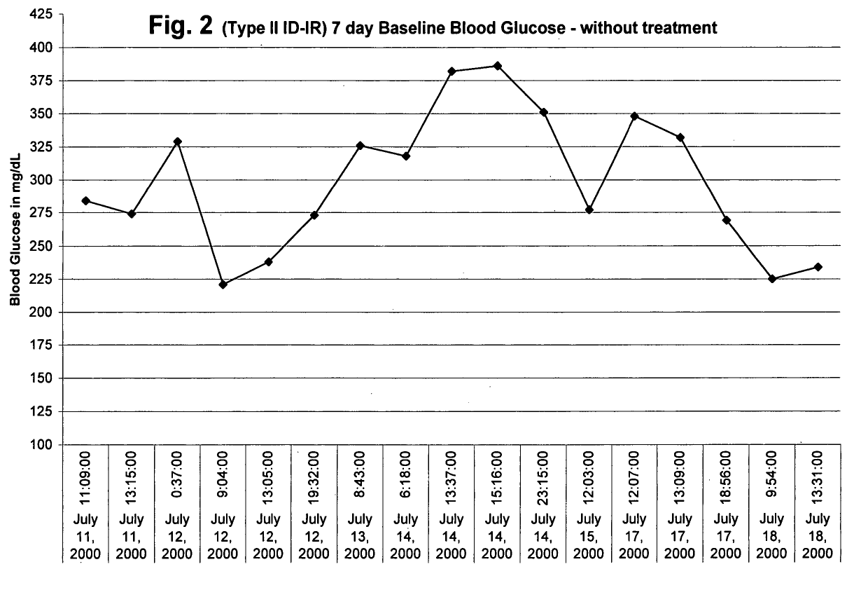

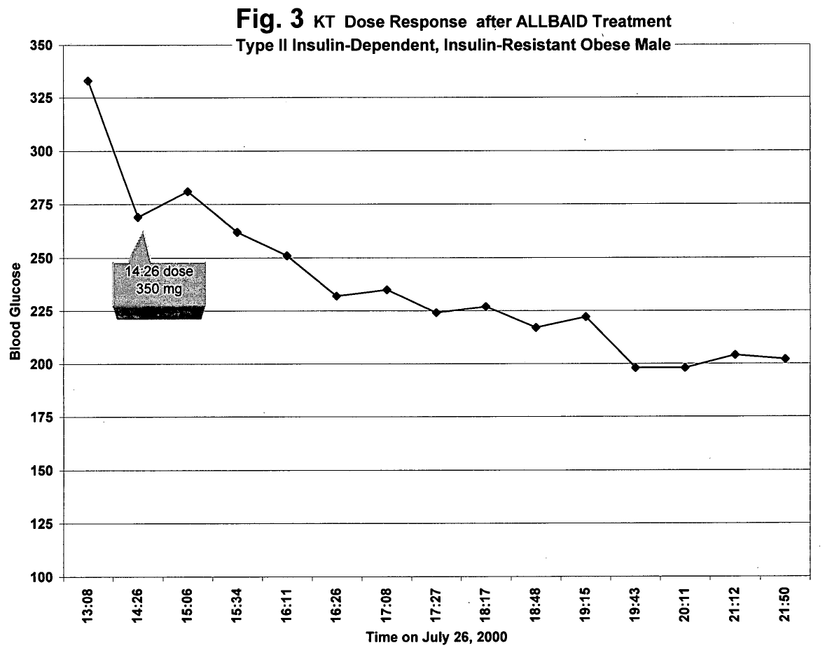

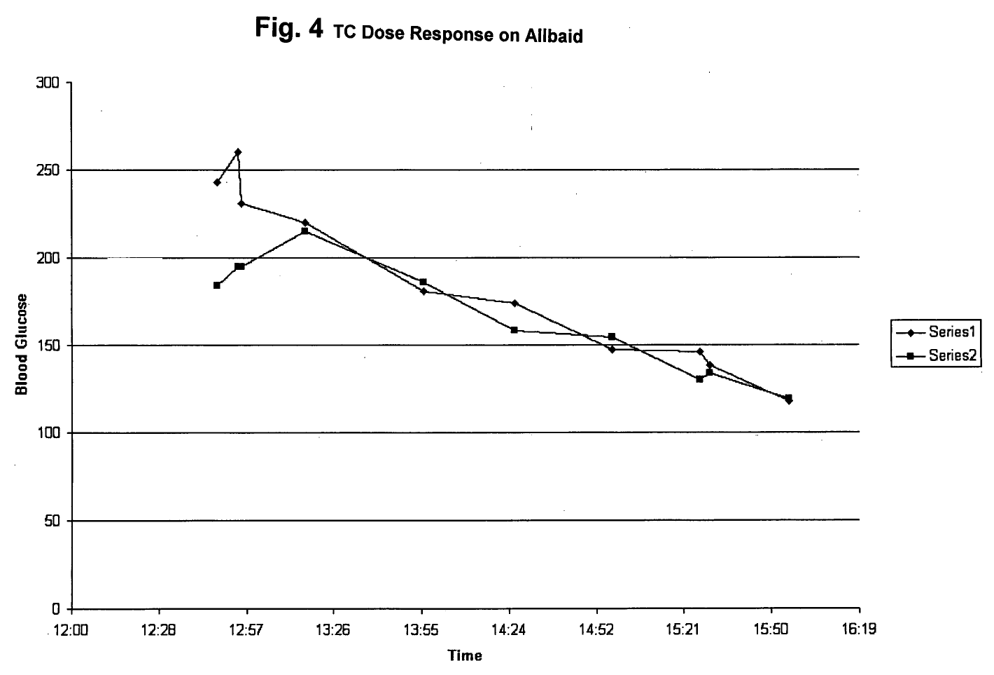

KT is a Type 11 insulin resistant morbidly obese male with a 10 year history of heart attacks due to diabetes and neuropathy. He is approximately 50 years old. KT was using 220 units of insulin per day with no drop in blood sugars or abatement of symptoms (see seven day base line in FIG. 2). Within 3 days of luteolin administration KT showed decreased neuropathy and normal nerve function was regained. Sensate and tactile functions returned even to peripheral extremities. Blood sugars dropped from 475 mg/dl (milliliters per deciliter) to 74 mg/dl in 19 days of luteolin use (FIG. 3). KT returned to work with reinstatement of insurance due to his doctor's assessment that he was no longer diabetic. His blood tests were normal and HbAlc was dropped by 5.9 points to near normal, from 13.9 to 8.0. TC, another male Type II diabetic, also showed a marked response to luteolin as shown in FIG. 4.

CL is a Type 1 seven year old boy. His father is a diabetic and a physician. After administration of luteolin CL decreased his insulin use and titrated completely off all insulin for 5 months. Blood tests came back completely normal according to his endoainologists. FA is a Type II diabetic who had lost spatial orientation and was unable to work or even conduct family time with his children and wife. He is approximately 40 years old. Within 30 days of luteolin usage in a formulation known as Setebaid, made of nonhypoglycemic materials, FA regained family participation, regained color and health, went back to work and now uses ⅕ of his former dosage amount of insulin per day. He maintains good and stable demeanor and relationships. DS is a Type II female in her mid forties. She had fatigue, deliriums and excess sugars in the 250 milliliters per deciliter range. After taking luteolin in the Setebaid formulation, with no other hypoglycemic materials, she regained energy, strength and was able to resume work on a full time basis. Her numbers fell to the mid one hundreds on a glucometer, which is in milliliter per deciliter of sugars in the blood.

Animal tests of luteolin were made at BRM (Biomedical Research Models, Inc.) an East coast contract research organization (CRO) that specializes in diabetes research. BRM performed research studies under confidentiality towards investigating the efficacy of a nutraceutical, Setebaid® (luteolin), using well-established genetic rodent models of Type 1 (BB/Wor) and Type II (BBZDR/Wor) diabetes. Historically, these strains have been widely used in similar pre-clinical studies to predict anti-diabetogenic efficacy.

The effect of luteolin treatment in chronic Type I diabetic rats was examined. In this study, lean male diabetics were randomly assigned to 3 treatment groups (3-4 rats/group). Each group received either: (1) 3 mg luteolin intragastrically; (2) a subcutaneous injection of PZI insulin (0.9-1.2 mU/day); or (3) no treatment. Blood glucose was evaluated from time 0 through 6 hours (11 AM-5 PM). The data were expressed as average blood glucose relative to time post treatment Rats that received a single injection of insulin showed a 75% decrease in blood glucose levels (415 to 112 mg/dl) within 6 hours of injection. This response was fully consistent with prior work in the Type I rat model. Rather remarkably, diabetic rats that received Setebaid® (luteolin) showed a 31% drop in blood glucose levels (445 to 307 mg/dl) in 6 hours. In comparison, there was no reduction in the hyperglycemic state in the control group over the same interval (414 to 404 mg/dl). Furthermore, no additive or synergistic effects were observed when both insulin and insulin treatments were given simultaneously. Thus, a single 3 mg dose of luteolin was able to reduce hyperglycemia within 6 hours as much as 31% in insulin-dependent diabetic (Type 1) rats.

Next, we evaluated the ability of luteolin treatment to reduce hyperglycemia in chronic Type 2 diabetic rats. This study, the dose and frequency of luteolin treatment was increased to compensate for the enhance metabolism of the obese rat. First, a 24 hour baseline study was performed on 9 chronic Type 2 rats. We found no significant change in hyperglycemia over this 24 hour period of analysis in the diabetic rats. Next, these same rats were randomly assigned to 3 groups and given various doses of luteolin at three times during the 24 hr period (11 AM, 2 PM and 8 PM). Blood glucose analysis was evaluated every 2 hours.

Rats that received the lowest dose of 50 mg three time a day (150 mg total) showed a 10.2% decrease in blood glucose levels within 24 hr period of treatment. In comparison, rats treated an intermediate dose of 150 mg (450 mg total) showed a 22.9% drop in blood glucose. Rats in the third group that received the highest dose of 250 mg (750 mg total) showed the greatest change in glucose, a 27.7% decrease. Interestingly, the intermediate dose given to one rat reduced its blood glucose 52% (777 to 372 mg/dl) within 18 hr of treatment. Unfortunately, that animal died sometime before the 24 hr time point as a result of an accidental perforation of the esophagus during the administration of drug. These results demonstrate that luteolin® treatment markedly reduced hyperglycemia in the Type II diabetic rats 10-28% over a 24 hour period, and that these observations were dose-dependent.

In the next experiment we elected to provide these same rats with a standardized dose over an extended period of treatment. This change in protocol resulted in further drop in blood glucose. The data were expressed for each rat as a percentage change in blood glucose level relative to each individual pre-treatment level.

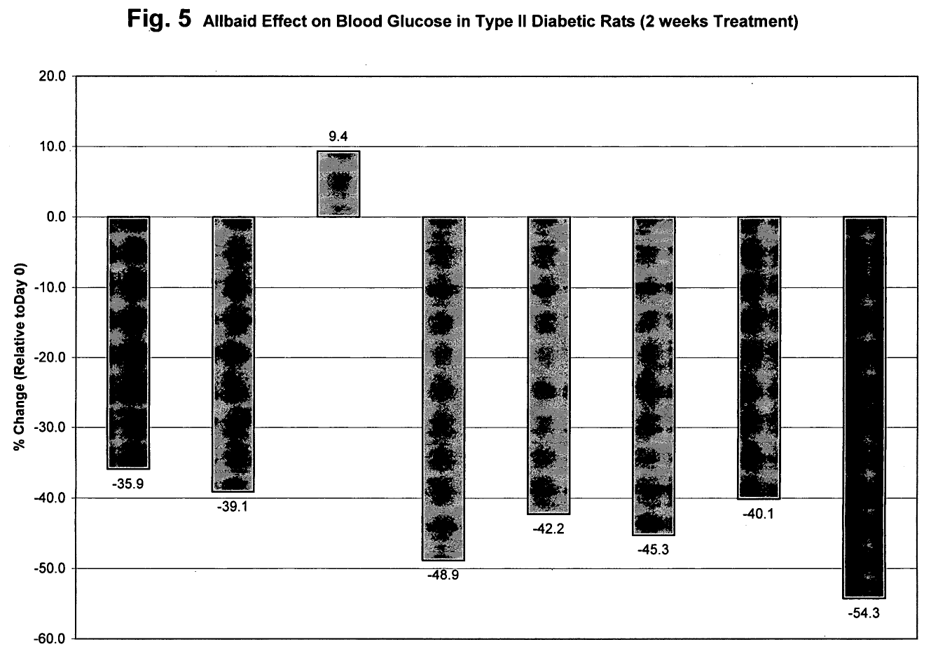

In FIG. 5, nearly all obese diabetic (Type II) rats treated with 50 mg (3×/day) for two weeks showed decreased blood glucose levels (range: 36% to 54%), excluding one rat. An esophageal fistula discovered at necropsy in the one rat showing a 9.3% increase in blood glucose likely prohibited effective dosing and response to treatment. Overall, blood glucose levels dropped an average of 41.1% (660 to 389 mg/dl) in the Type II diabetic rats.

These findings demonstrate that luteolin is a potent anti-diabetic agent that offers promise in the clinical setting.

I first discovered the luteolin effect after my experiments with herbal hypoglycemics. Several Brickellia californica live plants were located and harvested. Brickellia is a small to mid-sized shrub with relatively small, lobed leaves. Approximately four sprigs of leaves and stems were cut from the harvested plants. Each sprig was approximately 3 inches in length. The sprigs were placed in one half gallon of water and heated until boiling. Boiling continued for five minutes at which time, the extract was decanted from the container and cooled. The color of the decanted liquid was light brown. The cooled extract from the Brickellia californica sprigs was administered to four adult human males ranging from 30 to 40 years of age. Each of the males suffered from diabetes. The dosage to each subject was four to five glasses per day of the extract. Initially, all the subjects were self-administering insulin at a level 70 to 80 units per day. Blood glucose levels were measured periodically. After approximately three weeks, each of the subject's glucose levels began to drop. Consequently, the insulin administered to the subjects was decreased. After approximately six weeks all the subjects stop were able to control their diabetic conditions without the use of exogenous insulin.

These subjects suffered adult onset diabetes and were using insulin because ordinary anti-diabetic drugs proved ineffective. Presently, it is not know whether the Brickellia extract resulted in enhanced insulin production, in enhanced insulin function (e.g., higher number or more efficient insulin receptors) or in a lowering of blood sugar by some non-insulin mediated mechanism. The material appears to be equally effective in cases of insulin dependent diabetes. This may indicate that such diabetics have residual insulin production. Also, it is believed that continued inflammatory destruction (discussed above) of beta cells continues in insulin dependent diabetics. It appears likely that the Brickellia extract modulates this process allowing beta cell survival and insulin production. It is also possible that the extract also enhances the effect of residual insulin or operates by another, yet unknown, mechanism.

Live Bickellia californica plants were harvested and dried. The dried plant material was macerated using a mortar and pestle, transferred into a 125 ml Erlenmeyer flask and extracted with a mixture of chloroform and methanol in a ratio of 1:1. The mixture was stirred for four hours with a magnetic stirrer. The extract from the flask was then filtered to remove cellulosic debris and concentrated in a “rotavap” under a vacuum to yield a crude gummy residue. The residue was partitioned in chloroform and methanol to yield to two fractions labeled CHCl₃ (the more hydrophobic chloroform soluble fraction) and MeOH (the more hydrophilic methanol soluble fraction).

The CHCl₃ and MeOH fractions were analyzed using a Hewlett Packard 6890 gas chromatograph-mass spectrometer (GC-MS) fitted with an HP-5MS capillary column (30 meters×250 μm×0.25 μm). The analysis conditions were as follows: initial temperature was 125° C. which was held for five minutes, followed by an increase to 275° C. at a rate of 10° C. per minute with the final temperature of 275° C. being held 15 minutes. The analysis by the GC-MS of CHCl₃ fraction demonstrated the presence of a group of polar flavonoids with retention times in the range of 13-15 minutes, the presence of a group of sesquiterpenes with retention times between 16-18 minutes, and a small group of aliphatic hydrocarbons with retention times between 20-25 minutes. Analysis by GC-MS of the MeOH fraction produced similar results except that the MeOH fraction was largely free of the aliphatic hydrocarbons.

It is believed that the Brickellia californica extract includes the flavonoids dihydrokaemferol, apigenin, luteolin, myricetin and quercetin. Further, the many other species of Brickellia contain these, or similar flavonoids, albeit in different proportions, and should also be effective in treatment of diabetes. Experiments with diabetic test animals (rats and mice) were carried out. The Brickellia extract was effective in controlling blood glucose in these model systems. Further, the administration of synthetic versions of the Brickellia flavonoids were also effective at lowering glucose levels. In treatments involving a single flavonoid, luteolin was the most effective agent. However, there is some indication that a combination of luteolin with the other flavonoids, especially dihydrokaemferol and apigenin, results in an enhanced effect in that blood glucose can be maximally lowered with a lower overall flavonoid dose. The effect seems most pronounced when the molar concentration of luteolin is at least twice that of dihydrokaemferol and apigenin combined.

Whatever the route of flavonoid action, the results are not instantaneous. As explained above, Brickellia extract takes some weeks to maximally lower blood glucose. In animal models it takes several days for an appreciable lowering of blood glucose with the maximal effect requiring up to several weeks. This delay in results may explain why this effect has not been hitherto observed considering that many common fruits and vegetables contain flavonoids shown to be effective in the present invention. It would appear that sustained ingestion of adequate amounts of effective flavonoids is required. As an aside, it is well known that original human diets were rich in flavonoids whereas refined diets common in the industrialized nations are relatively flavonoid depauperate. Recent studies have suggested that the lack of dietary flavonoids is partially responsible for heart and vascular diseases. Now it appears that the worldwide “epidemic” of diabetes may also be a result of flavonoid starvation. Vegetarians are known to have lower incidences of diabetes as well as a number of other degenerate diseases. Conventional wisdom was that the lack of diabetes might be related to the relative absence of refined sugars from the vegetarian diet. An alternate explanation could well be the richness of flavonoids in these diets.

In addition to the equivalents of the claimed elements, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention.

1. An anti-diabetic composition comprising an aqueous extract of plants of the genus Brickellia.

2. The anti-diabetic composition of claim 1, wherein the extract is from Brickellia californica.

3. An anti-diabetic composition consisting of a flavonoid selected from the group consisting of luteolin, myricetin, dihydrokaemferol, apigenin, quercetin and mixtures thereof.

4. An anti-diabetic composition consisting of a mixture of luteolin, dihydrokaemferol and apigenin.

5. The anti-diabetic composition of claim 4, wherein the molar concentration of luteolin is at least twice that of dihydrokaemferol and apigenin added together.

6. A method for treatment of diabetes mellitus comprising the step of administering a quantity of an aqueous extract of plants of the genus Brickellia to result in a reduction in blood glucose.

7. The method of claim 6, wherein the extract is from Brickellia californica.

8. A method for treatment of diabetes mellitus consisting of the step of administering a quantity of a flavonoid selected from the group consisting of luteolin, myricetin, dihydrokaemferol, apigenin, quercetin and mixtures thereof to result in a reduction in blood glucose.

9. The method of claim 8, wherein a mixture of luteolin, dihydrokaemferol and apigenin is administered.

10. The method of claim 9, wherein the molar concentration of luteolin is at least twice that of dihydrokaemferol and apigenin added together.

11. A method of controlling diabetes mellitus in a mammal comprising the step of administering to the mammal a molecule that binds to Kᵥ1.3 ion channels.

12. The method of claim 1 1, wherein the molecule is a flavonoid.

13. The method of claim 12, wherein the flavonoid is luteolin.

14. A method of controlling unwanted proliferation to T-cells in a mammal comprising the step of administering to the mammal a molecule that binds to Kᵥ1.3 ion channels.

15. A method of screening a group of compounds for anti-diabetic activity in a mammal comprising the step of determining which members of the group binds to and blocks Kᵥ1.3 ion channels, wherein the members binding to and blocking Kᵥ1.3 ion channels are selected as having potential anti-diabetic activity.

16. A method of screening a group of compounds for ability to suppress autoimmune responses in a mammal comprising the step of determining which members of the group binds to and blocks Kᵥ1.3 ion channels, wherein the members binding to and blocking Kᵥ1.3 ion channels are selected as having potential ability to suppress autoimmune responses.

17. A compound that contrails diabetes mellitus in a mammal characterized in that the compound binds to and blocks Kᵥ1.3 ion channels.
